(19) United States
(12) Patent Application Publication
(10) Pub. No.: US 2008/0200933 A1
(43) Pub. Date: Aug. 21, 2008

(54) Surgical devices and methods for forming an anastomosis between organs by gaining access thereto through a natural orifice in the body

(76) Inventors: Gregory J. Bakos, Mason, OH (US); William D. Fox, New Richmond, OH (US)

Correspondence Address: KIRKPATRICK & LOCKHART PRESTON GATES ELLIS LLP, 535 SMITHFIELD STREET, PITTSBURGH, PA, 15222, US

(21) Appl. No.: 11/706,460

(22) Filed: Feb. 15, 2007

(51) Int. Cl. A61B 17/11

(52) U.S. Cl. 606/153

(57) ABSTRACT

A surgical instrument for creating an anastomosis between two organs. In various embodiments, the instrument may include a hollow outer sleeve for creating a tool-receiving passage between a natural orifice in the patient and the surgical site. The tool receiving passage may be used to operably accommodate various surgical instruments such as an endoscope, a hole-forming instrument, and suction and aeration tubes for assisting in the formation of the anastomosis. The hollow outer sleeve may further be used to position a hollow anastomosis sleeve segment within two aligned holes in the organs. The anastomosis sleeve segment supports an inflatable distal receptacle that is inflated within one of the organs and a proximal inflatable receptacle that is inflated in the other organ to retain the anastomosis sleeve segment in position. Thereafter, the hollow outer sleeve is withdrawn from the patient leaving the hollow anastomosis sleeve segment in position between the two organs.

### CROSS-REFERENCE TO RELATED APPLICATIONS

The present application is related to the following commonly-owned U.S. patent application filed on even date herewith, entitled “Surgical Devices and Methods Using Magnetic Force To Form an Anastomosis” to William D. Fox, (END6105USNP/KLG No. 070030) the disclosure of which is hereby incorporated by reference in its entirety.

### FIELD OF THE INVENTION

The present invention relates, in general, to surgical devices for forming an anastomosis between organs and, more particularly, to devices that can be inserted through a natural orifice in the body and used to form an anastomosis between various gastrointestinal organs.

### BACKGROUND OF THE INVENTION

Access to the abdominal cavity may, from time to time, be required for diagnostic and therapeutic endeavors for a variety of medical and surgical diseases. Historically, abdominal access has required a formal laparotomy to provide adequate exposure. Such procedures which require incisions to be made in the abdomen are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

In the past, such surgical procedures were also employed to address various problems occurring in the jejunum (a portion of the small intestine). For example, such procedures were commonly employed to address blockages or strictures in the jejunum or to address diseases occurring therein. In some situations, it becomes necessary to create a Gastro-Jejunostomy an anastomosis between the stomach and the jejunum. An anastomosis is the bringing together or joining of two hollow structures. In addition to suffering from the above-mentioned limitations, current laparoscopic and endoscopic surgical techniques also fail to provide a convenient way for inserting a distal mass and are generally incapable of applying sufficient presence and forces to effect a clinically acceptable compression anastomosis.

Over the years, various types of devices have been developed for creating an anastomosis between hollow organs or vessels. For example, published U.S. Patent Application No. US2005/0192602A1 to Manzo discloses the use of a tubular body member that has a device known as an expandable onion formed on its distal end for forming an anastomosis between the urethral stump and the bladder neck.

Published U.S. Patent Application No. US2005/0165378A1 to Heinrich et al. discloses a device that includes an inner and outer sleeve wherein a first expandable anchor is mounted on the inner sleeve and a second expandable anchor is mounted to an outer sleeve. Once in position, the first expandable anchor is expanded in one vessel and the second expandable anchor is expanded in a second vessel. A biocompatible medical glue is then applied to the joined vessels prior to removal of the device.

None of the aforementioned devices are designed to accommodate a camera or the like for viewing the surgical site area or are capable of operable supporting devices that may be used to form the exitotomy and enterotomy in the organs at desired locations. Moreover, often times during these procedures, it may be desirable to apply suction to the desired target organ during the formation of the enterotomy or exitotomy. The prior devices, however, lack such ability. In addition, it may be desirable to flush the target area with fluid prior to commencing the surgery. The aforementioned devices further lack such capability.

Consequently a significant need exists for an alternative to conventional surgery that eliminates abdominal incisions and incision-related complications by combining endoscopic and laparoscopic techniques to diagnose and treat abdominal pathology.

There is a further need for a surgical device that can be introduced into the stomach through the mouth and used to form a clinically acceptable compression anastomosis between the stomach and the jejunum and facilitate the use of a variety of different surgical instruments and tools at the surgical site.

The foregoing discussion is intended only to illustrate some of the shortcomings present in the field of the invention at the time, and should not be taken as a disavowal of claim scope.

### SUMMARY

In one aspect of the invention, there is provided a surgical instrument for creating an anastomosis between two organs. In various embodiments, the instrument includes a hollow outer sleeve that has a distal end and a proximal end. The hollow outer sleeve defines a tool-receiving passage for receiving at least one surgical instrument therethrough and further has a distal pressure supply lumen and a proximal pressure supply lumen therein. A hollow anastomosis sleeve segment is removably supported on the hollow outer sleeve adjacent its distal end. The hollow anastomosis sleeve segment supports a distal inflatable receptacle thereon that is in fluid communication with the distal pressure supply lumen and a proximal inflatable supply receptacle that is in fluid communication with the proximal pressure supply lumen.

In another general aspect of various embodiments of the present invention there is provided a surgical instrument for creating an anastomosis between two organs. In various embodiments, the instrument includes a hollow outer sleeve that has a plurality of segments formed in a distal portion thereof that converge to form a substantially tapered distal end that can be selectively splayed open upon passage of an instrument therethrough. In at least one embodiment, an anastomosis sleeve segment can be removably supported on the hollow outer sleeve. The anastomosis sleeve segment can support a distal inflatable receptacle that is in fluid communication with a distal pressure supply lumen in the hollow outer sleeve and a proximal inflatable receptacle that is in fluid communication with a proximal pressure supply lumen in the hollow outer sleeve.

In still another general aspect of various embodiments of the present invention there is provided a method for forming an anastomosis between first and second organs in a patient. In various forms, the method may include forming a hollow tool-receiving passage extending from a natural orifice in the patient into the first organ. The method may further include supporting a hole-forming instrument within the hollow tool-receiving passage and forming a first hole through the first organ and forming a second hole through the second organ using the hole-forming instrument. The method may also include positioning a hollow anastomosis sleeve segment having a distal inflatable receptacle and a proximal inflatable receptacle thereon within the first and second holes such that the distal inflatable receptacle is positioned within the second organ and the proximal inflatable receptacle is positioned within the first organ. Thereafter, the method may include inflating the distal inflatable receptacle and the proximal inflatable receptacle to retain the hollow anastomosis sleeve within the first and second holes.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain various principles of the present invention.

FIG. 1 is a diagrammatical view illustrating the use of one embodiment of a surgical instrument of the present invention inserted through a patient's mouth and esophagus to create an anastomosis between the patient's stomach and jejunum;

FIG. 2 is a side view of a distal portion of surgical instrument embodiment of the present invention having the distal and proximal inflatable receptacles in an uninflated state;

FIG. 3 is a partial cross-sectional view of the distal portion of the surgical instrument embodiment depicted in FIG. 2, with the air/vacuum tube and endoscope inserted into a tool receiving passage of the surgical instrument;

FIG. 4 is another side view of the distal portion of the surgical instrument of FIGS. 2 and 3 with the distal and proximal inflatable receptacles each in an inflated state;

FIG. 5 illustrates a portion of patient's stomach and jejunum with an endoscope inserted through the distal end of the outer sleeve of various embodiments of the present invention;

FIG. 6 is another view of a portion of the patient's stomach and jejunum as depicted in FIG. 5 with the vacuum/air tube extended out of the distal end of the outer sleeve and in engagement with a portion of the stomach wall;

FIG. 7 is another view of a portion of the patient's stomach and jejunum depicted in FIG. 6 wherein suction has been applied to a portion of the stomach wall through the vacuum/air tube;

FIG. 8 is another view of a portion of the patient's stomach and jejunum depicted in FIG. 7 wherein a hole-forming device has punctured a portion of the stomach wall that is drawn into the vacuum/air tube to form an exitotomy therethrough;

FIG. 9 is another view of a portion of the patient's stomach and jejunum illustrated in FIG. 8 after the vacuum/air tube and endoscope have been drawn back into the outer sleeve and with the pointed distal end of the outer sleeve inserted through the exitotomy created through a portion of the stomach wall;

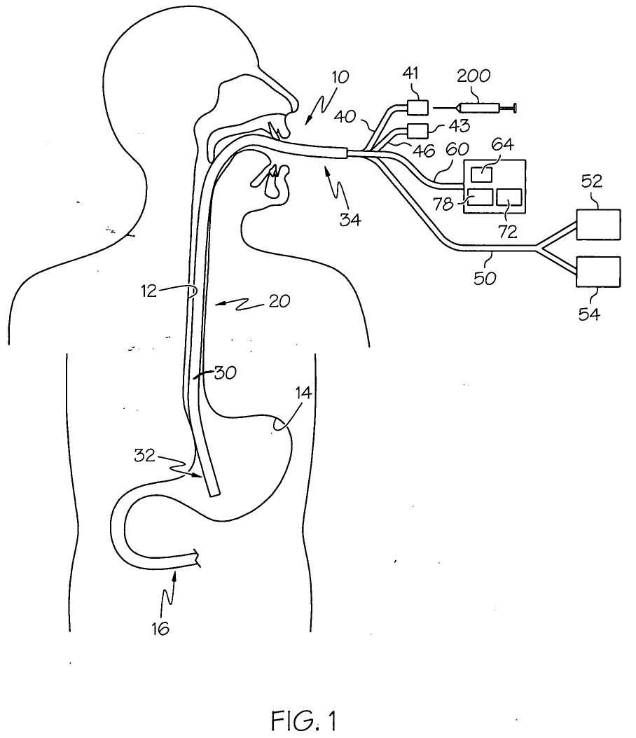

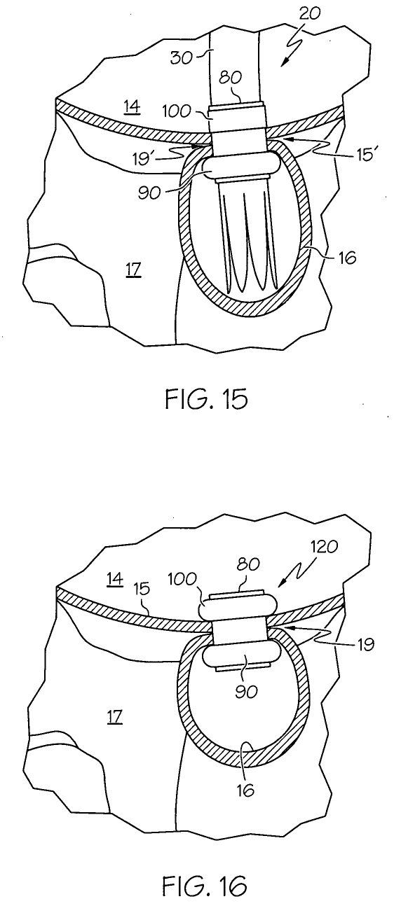

FIG. 1 0 is another view of a portion of the patient's stomach and jejunum illustrated in FIG. 9 wherein the endoscope has been advanced out through the distal end of the outer sleeve to view the peritoneum;

FIG. 11 is another view of a portion of the patient's stomach and jejunum illustrated in FIG. 10 wherein the endoscope has been used to select a target site for creating an enterotomy into the jejunum;

FIG. 12 is another view of a portion of the patient's stomach and jejunum illustrated in FIG. 11 wherein the vacuum/air tube is extended through the distal end of the outer sleeve and is applying a suction to the target portion of the jejunum wall;

FIG. 13 is another view of a portion of the patient's stomach and jejunum illustrated in FIG. 12 wherein the hole-forming device has punctured a portion of the jejunum wall that is drawn into the vacuum/air tube to form an enterotomy therethrough;

FIG. 14 is another view of a portion of the patient's stomach and jejunum illustrated in FIG. 13 after the vacuum/air tube and endoscope have been drawn back into the outer sleeve and with the pointed distal end of the outer sleeve inserted through the enterotomy created through a portion of the jejunum wall;

FIG. 15 is another view of a portion of the patient's stomach and jejunum illustrated in FIG. 14 wherein the distal inflatable receptacle has been inflated and the jejunum has been pulled into contact with the corresponding portion of the stomach; and

FIG. 16 is another view of a portion of the patient's stomach and jejunum illustrated in FIG. 15 with the proximal inflatable receptacle inflated and the outer sleeve assembly removed from the anastomosis sleeve.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

The present invention generally provides methods and devices for creating a Gastro-Jejunostomy via natural orifice procedures combined with a transorgan approach. While various exemplary embodiments are described herein for creating an anastomosis between the stomach and the small intestine by accessing those organs through the patient's mouth and esophagus, those of ordinary skill in the art will readily appreciate that unique and novel aspects of various embodiments of the present invention could successfully be employed in connection with forming anastomosis between other organs by gaining access thereto through other natural orifices such as, for example, the anus, the vagina, etc. without departing from the spirit and scope of the present invention.

It will be appreciated that the terms “proximal” and “distal” are used herein with reference to a clinician manipulating and end of the instrument 20 that protrudes out of the natural orifice. The term “proximal” referring to the portion closest to the clinician and the term “distal” referring to the portion located away from the clinician. It will be further appreciated that for convenience and clarity, spatial terms such as “vertical”, “horizontal”, “up” and “down” may be used herein with respect to the drawings. However, surgical instruments are used in many orientations and positions, and these terms are not intended to be limiting and absolute.

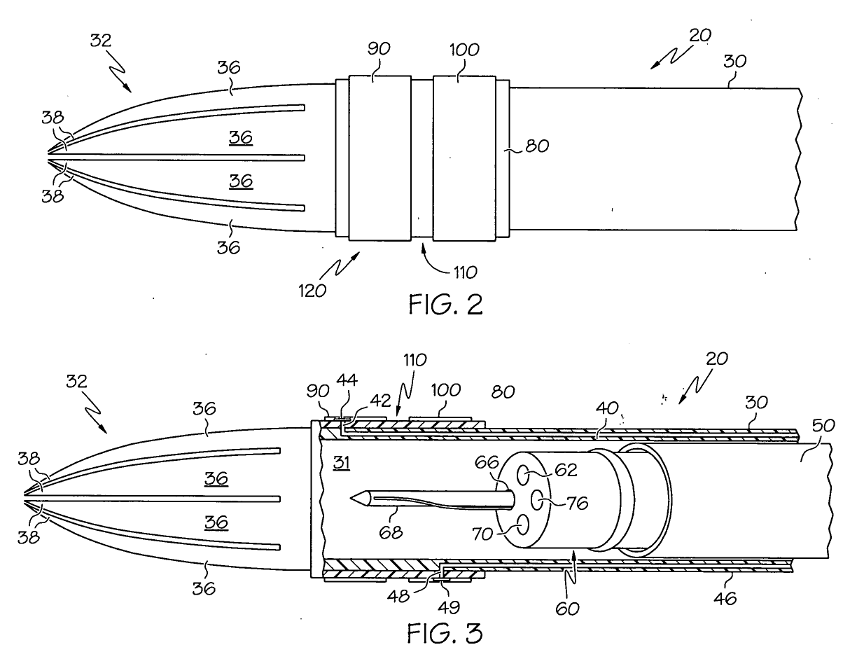

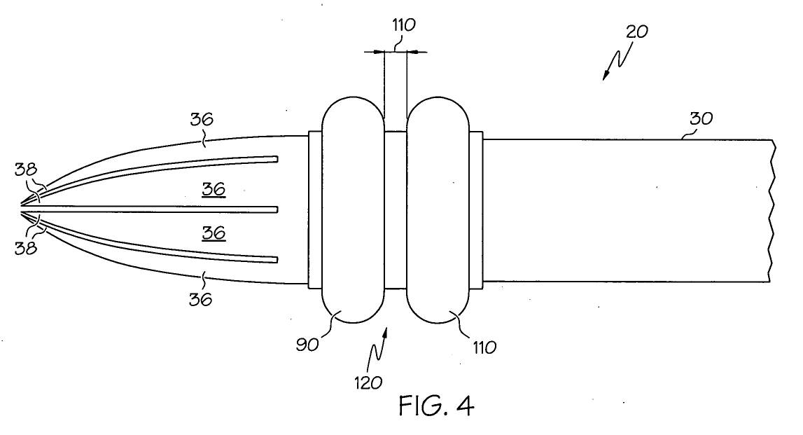

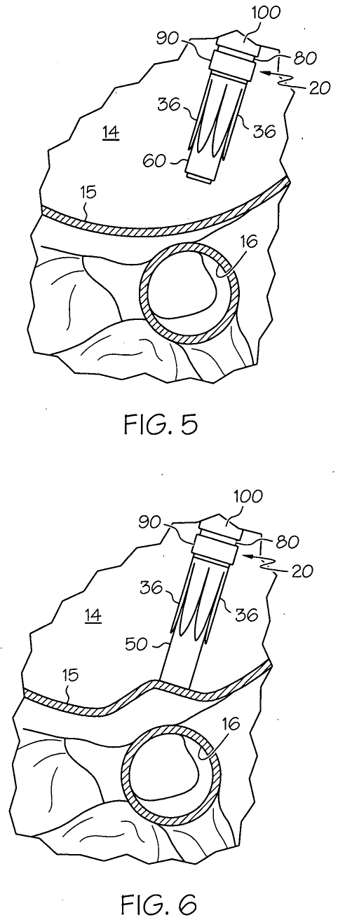

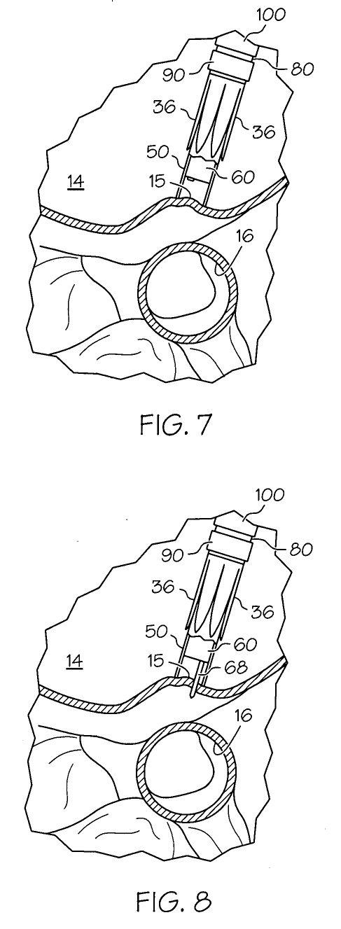

FIG. 1 illustrates, in general form, one exemplary surgical instrument 20 of the present invention that can be inserted through a natural orifice such as the mouth 10 and esophagus 12 into the stomach 14 to establish an anastomosis between the stomach 14 and the jejunum 16. As can be seen in FIGS. 2 and 3, the surgical instrument 20 may comprise a hollow outer sleeve 30 that has a distal end 32 and a proximal end 34 (FIG. 1). In various embodiments, the hollow outer sleeve 30 may be fabricated from, for example, nylon or high density polyethylene plastic. Referring to FIGS. 2 and 3, the distal end 32 of the hollow outer sleeve 30 may be formed with a plurality of tapered segments 36 that each have a substantially pointed distal end 38. In an unexpanded state (FIGS. 3 and 4) the distal ends 38 substantially converge together to provide the outer sleeve 30 with a substantially pointed distal end 32 and can flex open to permit other instruments to pass through the distal end 32 (FIG. 5). In various embodiments, the hollow outer sleeve 30 can serve to define a tool-receiving passage generally designated as 31 that extends from the natural orifice 10 to the surgical site.

As can be seen in FIG. 3, in various embodiments, one of the tools or surgical instruments that can be accommodated in the tool-receiving passage 31 is a hollow vacuum/air tube 50 that may communicate with at least one of a vacuum source 52 and a source of pressurized air 54 (FIG. 1). In at least one embodiment, the vacuum/air tube 50 can be sized to receive therein another surgical instrument in the form of an endoscope 60. A variety of different types of endoscopes are known and, therefore, their specific construction and operation will not be discussed in great detail herein. In various embodiments, the endoscope 60 may operably support a video camera 62 that communicates with a video display unit 64 (FIG. 1) that can be viewed by the surgeon during the operation.

In various embodiments, the endoscope 60 may further have one or more working channels 66 extending therethrough for receiving various instruments such as a hole-forming device 68, for example. The hole-forming device 68 may comprise, for example, a conventional Sphinctorotome, a needle knife or other incisor-type instrument that may be inserted through a working channel 66 in the endoscope 60. In addition, the endoscope 60 may further have a fluid-supply lumen 70 therethrough that is coupled to a source 72 of water, saline solution, etc. and/or an air supply lumen 76 that is coupled to the source of air 78. See FIGS. 1 and 3.

In various embodiments, an anastomosis sleeve 80 may be removably supported on the outer sleeve 30 adjacent the distal end 32. The anastomosis sleeve 80 can support a distal inflatable receptacle 90 and a proximal inflatable receptacle 100 that is spaced from the distal inflatable receptacle 90 to create a tissue compression zone 110 therebetween. The inflatable receptacles 90, 100 may each comprise an expandable balloon, pouch or bag that extends around, and is attached to, the anastomosis sleeve 80 by, for example, an adhesive such as cyanoacrylate, epoxy resin, or light activated glues such that a substantially fluid tight seal is established between each of the inflatable receptacles 90, 100 and the anastomosis sleeve 80. See FIG. 3. In various embodiments, the anastomosis sleeve 80 may be fabricated from, for example nylon or high density polyethylene plastic and the inflatable receptacles 90, 100 may each be fabricated from thin films of nylon, PET, polyurethane plastics or latex elastomers. In other embodiments, the inflatable receptacles 90, 100 may each be fabricated from a material that is not expandable, but nevertheless is sized to inflate into a desired shape as will be further described below. The anastomosis sleeve 80 and the inflatable receptacles 90, 100 are generally referred to herein collectively as the anastomosis assembly 120.

In various embodiments, as can be seen in FIG. 3, a distal pressure supply lumen 40 can be provided through the outer sleeve 30 and be placed in fluid communication with the distal inflatable receptacle 90 through a port 42 in the walls of sleeves 30, 80. A flexible distal check valve flap or sleeve 44 can be oriented over the port 42 to enable a pressurized fluid medium (e.g., air, water, saline, etc.) to flow into the distal inflatable receptacle 90 out of the distal pressure supply lumen 40 and cause the distal inflatable receptacle 90 to inflate on the anastomosis sleeve 80. See FIG. 4. In various embodiments, the check valve sleeve 44 may comprises a soft rubber or plastic sleeve that is constructed to permit the pressurized medium to enter the receptacle 90. A luer 41 may be coupled to the proximal end of the distal pressure supply lumen to enable the fluid medium to be injected therein by a conventional syringe 200, for example. See FIG. 1. After the receptacle 90 can be inflated to a desired shape, the flow of pressurized fluid medium into the receptacle 90 can be discontinued and the pressure in the distal supply lumen 40 may be relieved through its proximal end. Once the pressure inside the inflated receptacle 90 exceeds the pressure in the distal pressure supply lumen 40, the pressurized fluid medium within the receptacle 90 can cause the check valve sleeve 44 to prevent the pressurized fluid medium from flowing back out through the port 42.

Likewise, a proximal pressure supply lumen 46 can be provided through the outer sleeve 30 and be placed in fluid communication with the proximal inflatable receptacle 100 through a port 48 in the walls of sleeves 30, 80. A flexible proximal check valve flap or sleeve 49 can be oriented over the port 48 to enable a pressurized fluid medium (e.g., air, water, saline, etc.) to flow into the proximal inflatable receptacle 100 out of the proximal supply lumen 46 and cause the proximal inflatable receptacle 100 to inflate on the anastomosis sleeve 80. In various embodiments, the check valve flap 49 may comprises a soft rubber or plastic sleeve that is constructed to permit the pressurized medium to enter the receptacle 100. A luer 43 may be coupled to the proximal end of the proximal pressure supply lumen 46 to enable the pressurized fluid medium to be injected therein by a conventional syringe 200, for example. See FIG. 1. After the receptacle 100 is inflated to a desired shape, the flow of pressurized medium into the receptacle 100 can be discontinued. The pressure in the proximal pressure supply lumen 46 may then be relieved through its proximal end. Once the pressure inside the inflated receptacle 100 exceeds the pressure in the proximal pressure supply lumen 46, the pressurized medium within the receptacle 100 can cause the check valve sleeve 49 to prevent the pressurized medium from flowing back out through the port 48. As can be seen in FIG. 4, after the inflatable receptacles 90 and 100 have been inflated, a tissue compression zone 110 is formed therebetween. In at least one embodiment, the inflatable receptacles 90 and 100 can compress the walls of the organs together such that fluids in the organs do not escape from the organs through the holes created by the hole-forming device, for example. On the contrary, the anastomosis sleeve assembly can be configured such that such fluids in the organs pass through the sleeve 80 thereby creating a passage between the organs. In various embodiments, the tissue compression zone 110 may vary, for example, depending upon how full the receptacles 90 and 100 are inflated. Such distance however, may also be dependent upon the relative wall thicknesses of the organs through which the anastomosis assembly 120 is to be installed.

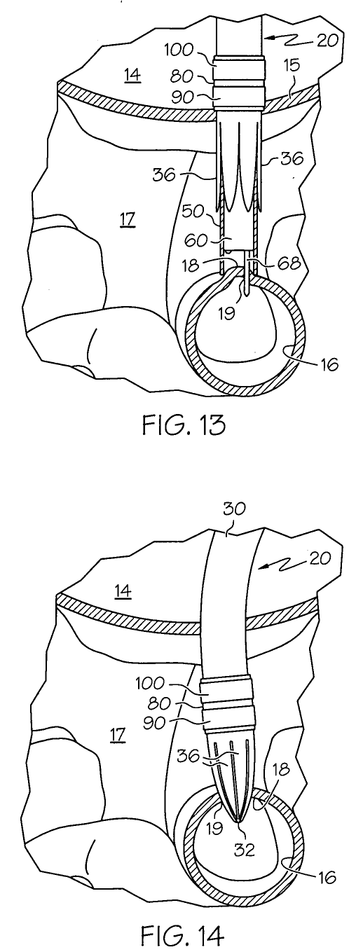

On exemplary method of using the surgical instrument 20 will now be described with reference to FIGS. 1, 3, and 5-16. To begin one method of the present invention for forming an anastomosis between two organs, the clinician may insert the outer sleeve 30 transorally through the patient's mouth 10 (FIG. 1) and esophagus 12 into the patient's stomach 14 to form a tool-receiving passage 31. In various embodiments, the patient's upper gastrointestinal track may be prepared utilizing known therapeutic techniques prior to insertion of the instrument 20. The outer sleeve 30 may also have the vacuum/air tube 40 and endoscope 60 therein upon insertion or those instruments may be inserted into the outer sleeve 30 after the distal end 32 of the outer sleeve 30 is positioned within the stomach 14. As the outer sleeve 30 and endoscope 60 are inserted into the stomach 14, the endoscope 60 is advanced out through the distal end 32 of the outer sleeve 30 causing the tapered segments 36 of the outer sleeve 30 to splay open as shown in FIG. 5. After the outer sleeve 30 and endoscope 60 have been positioned as shown in FIG. 5, the surgeon can select a desirable exitotomy site in the stomach wall 15 through use of the video camera 62 and display 64. After the desired exitotomy site has been located, the site may be flushed with water or other suitable fluid through the fluid supply lumen 70 in the endoscope 60 or through the vacuum/air tube 50.

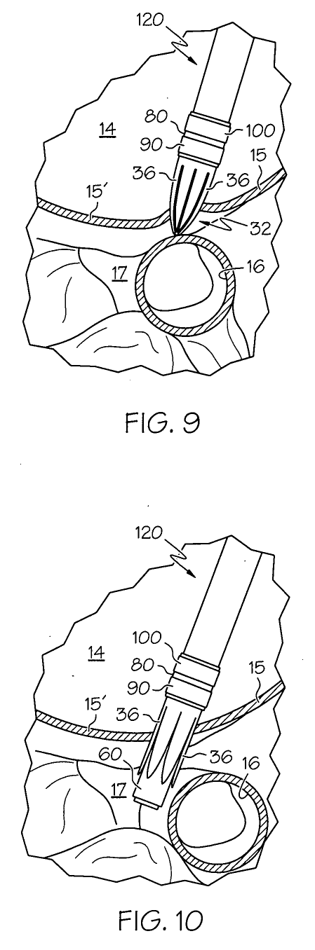

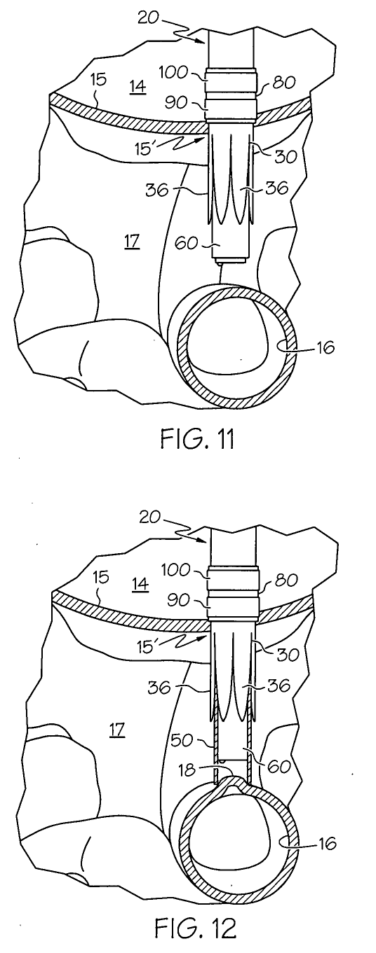

After the selected site has been flushed to minimize transfer of gastrointestinal contents, the vacuum/air tube 50 may then be advanced out through the distal end 32 of the outer sleeve 30 as shown in FIGS. 6 and 7. Suction may thereafter be applied to the vacuum/air tube 50 to draw a portion of the stomach wall 15 therein. Thereafter, the hole-forming tool 68 may be advanced out of the endoscope 60 to create a hole 15′, or exitotomy, through the stomach wall 15. After the exitotomy 15′ has been formed through the stomach wall 15, the vacuum/air tube 50 and endoscope 60 can be withdrawn into the outer sleeve 30 to enable the tapered segment portions 36 thereof to assume their unsplayed positions to form the pointed distal end 32 of the outer sleeve 30 as shown in FIG. 9. In at least one embodiment, the pointed distal end 32 of the outer sleeve 30 can facilitate entry of the outer sleeve 30 through the exitotomy 15′ and into the peritoneum 17.

After the distal end 32 of the outer sleeve 30 has been advanced through the exitotomy 15′, the endoscope 60 may be advanced out through the distal end 32 of the outer sleeve 30 as shown in FIG. 11 to enable the surgeon to view the second organ 16. In the example depicted in FIGS. 5-16, the second organ 16 comprises the jejunum. When in the position depicted in FIG. 11, the surgeon may blow air into the peritoneum 17 through the vacuum/air tube 50 or through a lumen in the endoscope 60 if desired to improve their view of the surgical field. After a enterotomy site has been selected on the second organ 16 utilizing the video camera 62 in the endoscope, the vacuum/air tube 50 may be advanced out through the distal end 32 of the sleeve 30. Suction may thereafter be applied to the vacuum/air tube 50 to draw the selected portion 18 of the jejunum 16 therein. Thereafter, the hole-forming tool 68 may be advanced out of the endoscope 60 to create a hole 19, or enterotomy, through the jejunum wall 18. See FIG. 13.

After the enterotomy 19 has been formed through the jejunum wall 18, the vacuum/air tube 50 and endoscope 60 may be withdrawn into the outer sleeve 30 to enable the tapered segment portions 36 thereof to assume their unsplayed positions to form the pointed distal end 32 of the outer sleeve 30 as shown in FIG. 14. The pointed distal end 32 of the outer sleeve 30 can facilitate entry of the outer sleeve 30 through the enterotomy 19 and into the jejunum 16. See FIG. 14. The distal end 32 of the outer sleeve 30 can then be advanced into the jejunum 16 until the distal inflatable receptacle 90 of anastomosis assembly 120 is received therein. Thereafter, pressurized fluid medium in the form of air, water, saline solution, etc. may be injected into the distal pressure supply lumen 40 by means of a syringe 200, for example. The pressurized fluid medium flows through the port 42 and under the one-way sleeve 44 into the inflatable distal receptacle 90. Once the inflatable distal receptacle 90 is sufficiently inflated to a desired state, the flow of pressurized fluid medium through the distal pressure supply lumen 40 can be discontinued and the pressure therein may be relieved such that the distal inflatable receptacle 90 remains inflated. See FIG. 15. As used herein, the term “desired state” with respect to the state of inflation of the inflatable receptacles 90, 100, means that the receptacles 90 and 100 have been sufficiently radially inflated to retain the hollow anastomosis sleeve segment 80 within the aligned first and second holes 15′ and 19. That is, the diameter of each the inflated receptacles 90 and 100 is sufficiently greater than the inner diameters of the first and second holes 15′, 19 so as to retain the anastomosis sleeve segment 80 therein. After the distal inflatable receptacle 90 has been inflated, the outer sleeve 30 and the anastomosis assembly 120 may be withdrawn in the proximal direction to pull the jejunum 16 against the stomach wall 15. Thereafter, the proximal inflatable receptacle 100 may be inflated by injecting pressurized fluid medium in the form of air, water, saline solution, etc. into the proximal pressure supply lumen 46 through the port 48 into the proximal inflatable receptacle 100 until the proximal inflatable receptacle 100 has been inflated to a desired shape. Thereafter, the flow of pressurized medium through the proximal pressure supply lumen 46 can be discontinued and the pressure therein may be relieved to enable the proximal inflatable receptacle 100 to remain inflated. See FIG. 16.

Once the receptacles 90, 100 have been inflated, portions of the stomach wall 15 and the jejunum wall 19 are compressed within the compression area 110 between the inflated receptacles 90, 100. After the anastomosis assembly 120 has been installed as shown in FIG. 16, the clinician may withdraw the outer sleeve 30 out of the anastomosis sleeve segment 80. The inner diameter of the anastomosis sleeve segment 80 may be sized relative to the outer diameter of the outer sleeve 30 such that the anastomosis sleeve segment 80 is able to be retained in position on the outer sleeve 30 through the above-mentioned procedures, yet enable the outer sleeve 30 to be withdrawn therefrom without affecting the position of the anastomosis assembly 120 once installed. After a period of healing, the clinician may reenter the patient's stomach 12 through the patient's mouth 10 and esophagus 12 with the endoscope 60 to examine the results and if desired, utilize appropriate instruments to collapse or puncture the receptacles 90, 100 and remove the anastomosis assembly 120

In alternative embodiments, one or both of the inflatable receptacles 90 and 100 may be replaced with a mechanically expandable structure known in the art as an “onion sleeve”. In general, an onion sleeve includes a substantially tubular body that has a plurality of ribs that define a plurality of longitudinally oriented slots. These structures generally remain in a collapsed state until the one of the ends thereof is axially advanced toward the other end to thereby cause at least a portion of the ribs to move radially outward. Such devices could be operably mounted on the anastomosis sleeve segment 80 to retain the sleeve segment 80 within the aligned first and second holes 15′, 19.

As can be readily appreciated from the foregoing, the various embodiments of the present invention described above represent a vast improvement over prior devises used to form an anastomosis between two organs or vessels. In particular, the various embodiments of the present invention are particularly well-suited for forming an anastomosis between two organs or vessels in the body by gaining access to those organs through a natural orifice in the patient. In addition, the unique and novel features of the various embodiments of the present invention may be used to form a tool-receiving passage from the natural orifice to the surgical site to enable a variety of different surgical tools to be inserted through the natural orifice and be used to assist with the installation of the anastomosis assembly.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art. Those of ordinary skill in the art will readily appreciate the different advantages provided by these various embodiments. While the various surgical instruments have been herein described in connection with the formation of a Gastro-Jejunostomy through a patient's mouth, those of ordinary skill in the art will readily appreciate that the unique and novel features of the various embodiments of the present invention may be effectively employed in connection with forming an anastomosis between other organs which may be accessed through other natural orifices in the patient. In addition, it is conceivable that the various embodiments of the present invention could have utility in some laparoscopic surgical procedures and therapies.

While several embodiments of the invention have been described, it should be apparent, however, that various modifications, alterations and adaptations to those embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include an combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The invention which is intended to be protected is not to be construed as limited to the particular embodiments disclosed. The embodiments are therefore to be regarded as illustrative rather than restrictive. Variations and changes may be made by others without departing from the spirit of the present invention. Accordingly, it is expressly intended that all such equivalents, variations and changes which fall within the spirit and scope of the present invention as defined in the claims be embraced thereby.

What is claimed is:

1. A surgical instrument for creating an anastomosis between two organs, comprising:
a hollow outer sleeve having a distal end and a proximal end and defining a tool-receiving passage therein for receiving at least one surgical instrument therethrough, said hollow outer sleeve further having a distal pressure supply lumen and a proximal pressure supply lumen extending therethrough; and
a hollow anastomosis sleeve segment removably supported on said hollow outer sleeve adjacent said distal end thereof, said hollow anastomosis sleeve segment supporting a distal inflatable receptacle thereon in fluid communication with said distal pressure supply lumen and a proximal inflatable supply receptacle thereon in fluid communication with said proximal pressure supply lumen.

2. A surgical instrument according to claim 1 further comprising:
a distal one way valve between said distal inflatable receptacle and said distal pressure supply lumen; and
a proximal one way valve between said proximal inflatable receptacle and said proximal pressure supply lumen.

3. A surgical instrument according to claim 1 wherein said at least one surgical instrument is selected from the group of surgical instruments consisting of a camera, a hole-forming instrument, a suction tube, an aeration tube, and a fluid supply lumen.

4. A surgical instrument according to claim 3 wherein at least one said surgical instrument includes a camera and a hole-forming instrument, and wherein said camera and said hole-forming instrument are operably supported within an endoscope received within said tool-receiving passage.

5. A surgical instrument according to claim 1 wherein said distal end of said outer sleeve comprises a plurality of flexible segments that are movable between a closed position wherein the flexible segments converge to form a substantially pointed end and an open position wherein the flexible segments are splayed outwardly.

6. A surgical instrument according to claim 1 wherein said distal inflatable receptacle and said proximal inflatable receptacle are inflated with a pressurized fluid selected from the group of pressurized fluids consisting of pressurized water, pressurized air, and pressurized saline solution.

7. A method for processing an instrument for surgery, the method comprising:
obtaining the surgical instrument of claim 1;
sterilizing the surgical instrument; and
storing the instrument in a sterile container.

8. A surgical instrument for creating an anastomosis between two organs comprising:
a hollow outer sleeve having a plurality of segments formed in a distal portion thereof that converge to form a substantially tapered distal end that can be selectively splayed open upon passage of an instrument therethrough; and
an anastomosis sleeve segment removably supported on said hollow outer sleeve, said anastomosis sleeve segment supporting a distal inflatable receptacle thereon in fluid communication with a distal pressure supply lumen in said hollow outer sleeve and a proximal inflatable receptacle thereon in fluid communication with a proximal pressure supply lumen in said hollow outer sleeve.

9. A surgical instrument according to claim 8 wherein said hollow outer sleeve defines a tool-receiving passage for receiving at least one surgical instrument therethrough.

10. A surgical instrument according to claim 9 wherein said at least one surgical instrument is selected from the group of surgical instruments consisting of a camera, a hole-forming instrument, a suction tube, an aeration tube, and a fluid supply lumen.

11. A surgical instrument according to claim 10 wherein said at least one surgical instrument includes a camera and a hole-forming instrument, and wherein said camera and said hole-forming instrument are operably supported within an endoscope received within said tool-receiving passage.

12. A method for forming an anastomosis between a first organ and a second organ in a patient, comprising:
forming a hollow tool-receiving passage extending from a natural orifice in the patient into the first organ;
supporting a hole-forming instrument within the hollow tool-receiving passage;
forming a first hole through a wall of the first organ using the hole-forming instrument;
forming a second hole through a wall of the second organ using the hole-forming instrument;
positioning a hollow anastomosis sleeve segment having a distal inflatable receptacle and a proximal inflatable receptacle thereon within the first and second holes such that the distal inflatable receptacle is positioned within the second organ and the proximal inflatable receptacle is positioned within the first organ;
inflating the distal inflatable receptacle and the proximal inflatable receptacle to retain the hollow anastomosis sleeve within the first and second holes.

13. A method according to claim 12 wherein said forming a hollow-tool receiving passage comprises passing a hollow sleeve having a hollow tool-receiving passage through the natural orifice into the first organ.

14. A method according to claim 13 further comprising supporting a camera within the tool-receiving passage.

15. A method according to claim 14 further comprising viewing the first organ with the camera to select a portion of the first organ to receive the first hole prior to said forming the first hole through the first organ.

16. A method according to claim 14 further comprising:
inserting the camera through the first hole in the first organ; and
viewing the second organ to select a portion of the second organ to receive the second hole prior to said forming a second hole through said second organ.

17. A method according to claim 12 further comprising applying suction to a portion of the first organ prior to forming the first hole therethrough.

18. A method according to claim 12 further comprising applying suction to a portion of the second organ prior to forming the second hole therethrough.

19. A method according to claim 12 wherein said positioning a hollow anastomosis sleeve segment comprises:
removably supporting the hollow anastomosis sleeve segment on a distal end portion of the hollow sleeve; and
manipulating the hollow sleeve to insert the hollow anastomosis sleeve segment into the first and second holes.

20. A method according to claim 19 wherein said inflating the distal inflatable receptacle and the proximal inflatable receptacle comprises:
injecting a pressurized fluid medium through a distal supply lumen in the hollow sleeve into the distal inflatable receptacle; and
injecting a pressurized fluid medium through a proximal supply lumen in the hollow sleeve into the proximal inflatable receptacle.

21. A method according to claim 20 further comprising withdrawing the hollow sleeve out of the hollow anastomosis sleeve segment after the distal inflatable receptacle and the proximal inflatable receptacle have been inflated.
